(19) United States
(12) Patent Application Publication
(10) Pub. No.: US 2012/0101380 A1
(43) Pub. Date: Apr. 26, 2012

(54) MEDICAL INSTRUMENT

(75) Inventors: Iris Blum, Fuldabruck (DE); Martin Sippel, Melsungen (DE)

(73) Assignee: B. Braun Melsungen AG, Melsungen (DE)

(21) Appl. No.: 13/291,210

(22) Filed: Nov. 8, 2011

Related U.S. Application Data
(63) Continuation of application No. PCT/EP2010/056281, filed on May 7, 2010.

(30) Foreign Application Priority Data
May 8, 2009 (DE) 10 2009 020 893.3

(51) Int. Cl. A61B 8/00

(52) U.S. Cl. 600/437

(57) ABSTRACT

To further improve, under ultrasonic observation, the visibility of a medical instrument with at least one portion of the instrument that can be inserted into body tissue of a patient and has a surface structure reflecting ultrasound waves, wherein the surface structure comprises a number of reflection elements, it is proposed that at least three, and at most nine, reflection elements which are arranged in a defined way in relation to one another define a reflection element group and that the surface structure comprises at least two reflection element groups.

### CROSS-REFERENCE TO RELATED APPLICATIONS

This application is a continuation application of International Application No. PCT/EP2010/056281 filed May 7, 2010, which claims priority to German Application No. 10 2009 020 893.3 filed May 8, 2009, the contents of both applications being incorporated herein by reference in their entirety and for all purposes.

### FIELD OF THE INVENTION

The present invention relates to medical instruments generally, and more specifically to a medical instrument with at least one instrument portion which can be inserted into body tissue of a patient and has a surface structure reflecting ultrasound waves, the surface structure comprising a plurality of reflection elements.

### BACKGROUND OF THE INVENTION

Medical instruments of the type mentioned at the outset may have one, two, three or more instrument portions and are used to treat patients, for example in the form of cannulas or endoscopic instruments. It is frequently very important to recognise the precise position and optionally also an orientation of the instrument inside the patient's body. One possibility for determining the position and/or orientation of the at least one instrument portion in the patient's body is to make the at least one instrument portion visible by means of ultrasound. In particular in the case of very small diameters of the at least one instrument portion, however, the latter can only be poorly seen, or not at all, under ultrasonic observation. It has therefore already been proposed to provide the at least one instrument portion with a surface structure comprising a plurality of reflection elements. A medical instrument with a surface structure of this type is known, for example, from U.S. Pat. No. 6,053,870. However, the at least one instrument portion can only be made visible to a limited extent by the known surface structure.

### SUMMARY OF THE INVENTION

In accordance with the invention a medical instrument with at least one instrument portion, which can be inserted into body tissue of a patient, has a surface structure reflecting ultrasound waves. The surface structure comprises a plurality of reflection elements. At least three and a maximum of nine reflection elements, which are arranged in a defined manner relative to one another, define a reflection element group. The surface structure comprises at least two reflection element groups.

### BRIEF DESCRIPTION OF THE DRAWING FIGURES

The foregoing summary and the following description may be better understood in conjunction with the drawing figures, of which:

FIG. 1: shows a perspective overall view of a medical instrument;

FIG. 2: shows a perspective view of a distal end of an instrument portion of the instrument shown in FIG. 1, which can be inserted into body tissue of a patient;

FIG. 3: shows an enlarged plan view of a partial region of the instrument portion shown in FIG. 2, comprising a reflection element group;

FIG. 4a: shows a sectional view along the line 4a-4a in FIG. 3;

FIG. 4b: shows a sectional view along the line 4b-4b in FIG. 3;

FIG. 4c: shows a sectional view along the line 4c-4c in FIG. 3;

FIG. 4d: shows a view analogous to FIG. 4c of an alternative embodiment of a reflection element;

FIG. 5: shows a sectional view analogous to FIG. 4a of an alternative embodiment of a reflection element;

FIG. 6: shows a plan view of a further alternative embodiment of a reflection element;

FIG. 7: shows a sectional view along the line 7-7 in FIG. 6;

FIG. 8a: shows a side view of a distal end of a further embodiment of a medical instrument;

FIG. 8b: shows a sectional view along the line 8b-8b in FIG. 8a;

FIG. 8c: shows a side view in the direction of the arrow A of the embodiment schematically shown in FIG. 8a;

FIG. 9a: shows a side view of a distal end of a further embodiment of a medical instrument;

FIG. 9b: shows a sectional view along the line 9b-9b in FIG. 9a; and

FIG. 9c: shows a side view in the direction of the arrow B of the embodiment schematically shown in FIG. 9a.

### DETAILED DESCRIPTION OF THE INVENTION

Although the invention is illustrated and described herein with reference to specific embodiments, the invention is not intended to be limited to the details shown. Rather, various modifications may be made in the details within the scope and range of equivalents of the claims and without departing from the invention.

The present invention relates to a medical instrument with at least one instrument portion, which can be inserted into body tissue of a patient and has a surface structure reflecting ultrasound waves, the surface structure comprising a plurality of reflection elements, wherein at least three and a maximum of nine reflection elements, which are arranged in a defined manner relative to one another, define a reflection element group and wherein the surface structure comprises at least two reflection element groups.

A surface structure optimised in the proposed manner increases the visibility of the at least one instrument portion under ultrasonic observation significantly. The configuration of reflection element groups by means of three to nine reflection elements allows substructures of the surface structure with optimised visibility to be found and to be defined. A view of the at least one instrument portion under ultrasonic observation can easily be improved by a corresponding arrangement of two or more reflection element groups of this type to facilitate the finding of a position and/or an orientation of the at least one instrument portion in the body of a patient. The visibility of the reflection element groups is improved, in particular, in that owing to the individual reflection elements, as a whole, a greater boundary line or a greater length thereof compared to only one single reflection element can be produced, so reflectivity for the ultrasound waves can be improved even with a very small surface. By means of a corresponding choice of the size and arrangement of the reflection elements, it can be achieved that, overall, an image point, which is also called a “pixel”, and is enlarged by these together, becomes visible under ultrasonic observation or the reflection elements remain recognisable individually.

In a particularly simple way, a reflection element group nevertheless becomes well visible under ultrasonic observation if it is defined by three reflection elements. An enlarged image point, which is also called a “pixel”, can thus be produced overall by a corresponding arrangement under ultrasonic observation by means of the reflection elements forming the reflection element group. If the reflection elements are sufficiently large and spaced far enough apart from one another, they can also be made visible separately by ultrasound irradiation. Furthermore, the reflection element group with three reflection elements can also be configured in such a way that it can itself already display an orientation of the at least one instrument portion.

The medical instrument can be produced particularly easily if all the reflection element groups are identically configured. They are then visible in an identical manner under ultrasound monitoring.

Each reflection element group advantageously comprises at least two reflection elements, which are arranged offset with respect to one another in the longitudinal direction of the instrument portion. In particular, the two, three or even more reflection elements arranged offset can be arranged offset parallel to a longitudinal axis of the instrument portion. Furthermore, they can also be additionally arranged offset in the peripheral direction relative to one another. Depending on the positioning, a longitudinal direction defined by the at least one instrument portion can thus become visible to an optimised extent under ultrasound monitoring.

To provide defined structures which are visible to an optimised extent by ultrasound, it can also be advantageous if the at least two reflection elements arranged offset with respect to one another in the longitudinal direction of the instrument portion are different in size and/or are differently formed. Different in size can, in particular, mean that an area of the instrument portion surface covered by them is of a different size. Furthermore, mutually geometrically similar forms of the reflection elements arranged offset, which, however, have surface areas of the at least one instrument portion of different sizes, are conceivable.

The medical instrument becomes particular easy to produce if the at least two reflection elements arranged offset with respect to one another in the longitudinal direction of the instrument portion are identically configured.

Furthermore, it may be advantageous if a spacing in the longitudinal direction between two reflection elements of a reflection element group arranged offset with respect to one another in the longitudinal direction of the instrument portion corresponds to Y-times a length of the smaller of the reflection elements in the longitudinal direction and that Y has a value in the range from 0.5 to 8. Providing reflection elements in the given spacing from one another in particular allows corresponding structures to be recognised under ultrasonic observation still with adequately good resolution.

Y advantageously has a value in the range from 2 to 5. A spacing defined in this manner allows the structures of individual reflection elements to be introduced in an optimised manner into a total structure of a reflection element group, which can be recognised under ultrasonic observation.

According to a further advantageous embodiment of the invention, it may be provided that a reflection element group spacing in the longitudinal direction between two reflection element groups arranged offset with respect to one another in the longitudinal direction of the instrument portion corresponds to Z-times a length of the smallest of the reflection elements in the longitudinal direction and that Z has a value in the range from 0.5 to 8. A spacing of the reflection element groups from one another in the given range makes it possible to recognise the individual reflection element groups under ultrasonic observation reliably separated from one another.

Z advantageously has a value in the range from 2 to 5. Corresponding spacing ratios make it possible to recognise the reflection element groups in an optimised manner and clearly separated from one another under ultrasonic observation.

It is advantageous if each reflection element group comprises at least two reflection elements, which are arranged offset in the peripheral direction transverse to the longitudinal direction of the instrument portion. This configuration has the advantage that each reflection element group, even if the at least one instrument portion is rotated about its longitudinal axis, can be seen well in the ultrasound image. Two, three, four or more reflection elements can be arranged offset in the peripheral direction.

The configuration of the medical instrument becomes particularly simple if the at least two reflection elements arranged offset in the peripheral direction transverse to the longitudinal direction of the instrument portion are identically configured.

It is advantageous if each reflection element is mirror-symmetrical with respect to a mirror plane containing a longitudinal axis of the instrument portion. Reflection elements of this type can be produced particularly easily. Furthermore, because of their symmetry even under corresponding conditions, they can display an orientation of the at least one instrument portion.

Each reflection element group is advantageously mirror-symmetrical with respect to a mirror plane containing a longitudinal axis of the instrument portion. With a corresponding arrangement of the reflection elements of the reflection element group, an orientation of the at least one instrument portion can thus be easily and reliably detected, in particular under ultrasonic observation.

Basically, the reflection element groups of the surface structure could be arranged in any desired manner. However, the surface structure is advantageously mirror-symmetrical as a whole with respect to a mirror plane containing a longitudinal axis of the instrument portion. It is thus possible for an orientation and position of the at least one instrument portion to be displayed directly to an operator of the instrument under ultrasonic observation.

It can easily be made possible for an operator to observe an orientation, for example a rotational position of the at least one instrument portion about the longitudinal axis thereof under ultrasound monitoring if the surface structure as a whole extends in the peripheral direction in relation to a longitudinal axis of the instrument portion over an angle range of a maximum of 180°. By rotating the at least one instrument portion through 180°, the latter can therefore be made visible or invisible by ultrasound. In particular, it is possible because of the proposed configuration to clearly disclose an orientation, for example of a distal end of the instrument portion that is non-rotationally symmetrically formed, by means of the surface structure. In particular, reflection elements arranged offset in the peripheral direction can define the angle range. With a corresponding arrangement of the reflection elements arranged offset in the peripheral direction relative to a reflection element arranged offset in the longitudinal direction, the latter can be made even more clearly visible under ultrasonic observation. In particular, an individual reflection element arranged offset in the longitudinal direction can also thus be made more visible under ultrasound.

The angle range is advantageously a maximum of 160°. A limitation to this angle range allows a still further optimised visibility of the surface structure to determine an orientation of the at least one instrument portion. It is favourable if the angle range is at least 50° and a maximum of 130°.

Particularly good visibility of the surface structure can be achieved if the reflection elements extend in each case over a reflection element angle range of about 5° to about 80° in the peripheral direction based on a longitudinal axis of the instrument portion. Thus, in particular depending on a diameter of the at least one instrument portion, adequately large structures can be provided by the reflection elements, which can ensure increased reflectivity to ultrasound.

The reflection element angle range advantageously has a value in a range from about 10° to about 70°. Thus, in particular two or even more reflection elements can be reliably separated from one another optically under ultrasonic observation, even if the latter are arranged offset in the peripheral direction in relation to the longitudinal axis of the at least one instrument portion.

In order, for example, to be able to form a cannula, it is advantageous if the at least one instrument portion is in the form of a hollow shaft. By means of the hollow shaft, in particular a channel can be formed, by means of which instruments can be inserted into a patient's body. Furthermore, a hollow shaft is also suitable to introduce fluids into a patient's body or remove them therefrom.

It is advantageous if the reflection elements are in the form of recesses and/or protrusions. These can be easily produced and by means of limit lines and/or limit faces, which are formed on the basis of the recesses or protrusions, with respect to the surface of the at least one instrument portion, allow increased reflectivity to ultrasound to be achieved.

In the case of a hollow shaft, in particular, in order to avoid it undesirably being perforated, it is advantageous if the shaft comprises a wall and if a height and/or a depth of the reflection elements in relation to a longitudinal axis of the instrument portion is smaller than a thickness of the wall. It is thus ensured that the wall of the shaft can be closed throughout.

To avoid a weakening of the shaft, it is furthermore advantageous if the height and/or the depth of the reflection elements is at most half as great as the thickness of the wall. Adequate reflectivity can thus also be ensured.

The height or the depth of at least one of the reflection elements advantageously varies parallel to the longitudinal direction of the instrument portion. Obviously, the height or the depth of all the reflection elements can be provided accordingly. It can thus be achieved, for example, that a reflectivity of the surface structure to ultrasound is particular great in certain advantageous directions.

Furthermore, it may be advantageous if the height or the depth of at least one of the reflection elements in the peripheral direction varies in relation to the longitudinal direction of the instrument portion. It is obviously also conceivable to design all the reflection elements in a corresponding manner. Owing to the variable height, a reflectivity of the reflection elements in certain regions thereof can be increased or reduced, which can be easily recognised under ultrasonic observation by an operator and can therefore be used for improved recognisability of the instrument portion.

According to a further advantageous embodiment of the invention, it may furthermore be provided that at least one reflection element of each reflection element group has an edge or side face extending transverse to a longitudinal direction of the instrument portion. As a result, in particular in a direction parallel or substantially parallel to the longitudinal direction of the instrument portion, a reflectivity of the surface structure can be maximised, so visibility of the instrument portion is particularly good under certain orientations.

In order to be able to particularly easily specify a clear orientation of a reflection element group visible under ultrasound, it is advantageous if at least one reflection element group comprises an odd number of reflection elements. Obviously, all the reflection element groups may also comprise an uneven number of reflection elements. In particular, three, five, seven or nine reflection elements may form a reflection element group.

Each reflection element group advantageously defines at least two reflection element planes spaced apart from one another in the longitudinal direction. These may be made visible to an optimised extent with a corresponding configuration and arrangement of reflection elements. In particular, rows of reflection elements extending transverse to the longitudinal direction, which intersect the reflection element planes, can thus be defined. The rows can, in particular, form structures completely or partially annularly surrounding the instrument portion.

At least one reflection element plane is advantageously defined only by one single reflection element. Other reflection element planes may, for example, also be defined by two or more reflection elements. Particularly sharp structures can thus be made visible under ultrasound.

The reflection element planes advantageously extend transverse to the longitudinal direction of the at least one instrument portion. Annular or partially annular structures, in particular, can thus be formed. Furthermore, because of the known spacings between the reflection element planes, spacings can thus also be easily and reliably determined under ultrasonic observation inside the body. The at least one instrument portion with one of the proposed surface structures is therefore also suitable as a measure for longitudinal measurements in body tissue.

A reflectivity of a reflection element can be further increased if a side boundary of a recess is bead-like and projects, at least in portions, slightly over an outer surface of the at least one instrument portion. It is also conceivable to make all the side boundaries of a recess bead-like in this form.

The instrument becomes particularly easy to produce if at least one reflection element of a reflection element group is in the form of a triangle. It is conceivable for two, three or all the reflection elements of a reflection element group to be in the form of triangles.

Triangles are excellently suitable because of their symmetry in order, already as a single reflection element, to make an orientation of the at least one instrument portion visible.

The triangle is advantageously in the form of an isosceles triangle. Thus, for example with an orientation of a plane of symmetry of the isosceles triangle parallel to a longitudinal direction of the at least one instrument portion, a direct inference of an orientation thereof can be made under ultrasound monitoring.

It is advantageous if a tip of the triangle points in the proximal or distal direction. Thus, under ultrasound monitoring, an orientation of the at least one instrument portion can be directly determined.

In order to make the at least one instrument portion visible under ultrasound in an optimal manner, it is advantageous if the number of reflection element groups is in a range from 3 to 25.

The number of reflection element groups is advantageously in a range from 7 to 15.

The medical instrument becomes particularly easy to produce if the reflection elements are formed by laser processing of the at least one instrument portion. In particular it can thus be avoided, in contrast to a pressing-in of the surface structure into the at least one instrument portion, that said instrument portion is squeezed in an undesired manner. In particular with the configuration of the at least one instrument portion in the form of a hollow shaft, it can thus be ensured that a channel defined by the shaft is not constricted by the configuration of the surface structure. Furthermore, a microstructure of the reflection elements, which can additionally increase reflectivity, can be provided by the laser processing.

In order to be able to insert the at least one instrument portion easily and reliably into body tissue, it is advantageous if it has a distal end which is in the form of a tip. In particular, the tip can define a tip plane inclined relative to a longitudinal direction defined by the instrument portion, for example by an end face of the tip.

According to the invention, the reflection element groups can directly adjoin the proximal end of the tip. However, it is particularly advantageous if a spacing between a proximal end of the tip and a distal end of a reflection element group provided adjacent to the tip corresponds to X-times the length of the tip and that X is in a range of 0.5 to 5. Depending on the configuration of the tip, the tip itself or a part thereof, can define a reflection element for ultrasound, for example an end face of the tip inclined in relation to a longitudinal axis of the instrument portion. A spacing in the given range makes it possible for the reflection element formed by the tip to be made visible clearly separated from the closest reflection element of the adjacent reflection element group under ultrasonic observation.

In this manner, a weakening of the at least one instrument portion in the region of the tip because of the configuration of the surface structure can be avoided. Despite this, a position of the tip can be easily and reliably determined by an operator of the instrument with knowledge of the spacing of the most distal reflection element group from the distal end of the tip.

X is advantageously in a range from 1.3 to 2. A spacing of this type makes it possible to precisely give the position of a distal end of the tip under ultrasonic observation and to simultaneously avoid a weakening of the tip.

The medical instrument advantageously comprises an instrument portion in the form of a cannula. The cannula can, in particular, be electrically conductive in order to allow the function of a stimulation cannula. Cannulas of this type are, in particular, suitable for use in anaesthesia.

The cannula is advantageously in the form of a stimulation cannula for nerve blocks. In this case, it can, in particular, be completely, partially and/or at points electrically conductive in order to stimulate body tissue, for example nerve paths by means of electrical signals, in particular currents.

In order to be able to carry out an electrical stimulation inside the body of the patient in a targeted manner, it is advantageous if the at least one instrument portion has an outer surface which comprises the surface structure reflecting the ultrasound waves, and which is provided with an electrically insulating layer. The layer channel can be in the form of a coating or in the form of a sheath or sleeve pushed onto the instrument portion.

The layer is advantageously produced from an insulation material. Electrical currents can therefore be conducted through the at least one instrument portion, for example through to a tip thereof, without the body tissue being able to be exposed to a current by contact with the instrument portion, with the exception of the tip.

The instrument becomes particularly easy and economical to produce if the insulation material is a plastics material or a ceramic. For example, a plastics material and likewise a ceramic can be sprayed onto the at least one instrument portion. Plastics materials can also, in particular, be applied by a bath coating.

Particularly high electric strength can be achieved if the plastics material is polytetrafluoroethylene (PTFE).

According to a further advantageous embodiment of the invention, it may furthermore be provided that the instrument comprises an electrical connection device to connect the instrument to a current or voltage source. The instrument can this be easily subjected to currents for introduction into a patient's body.

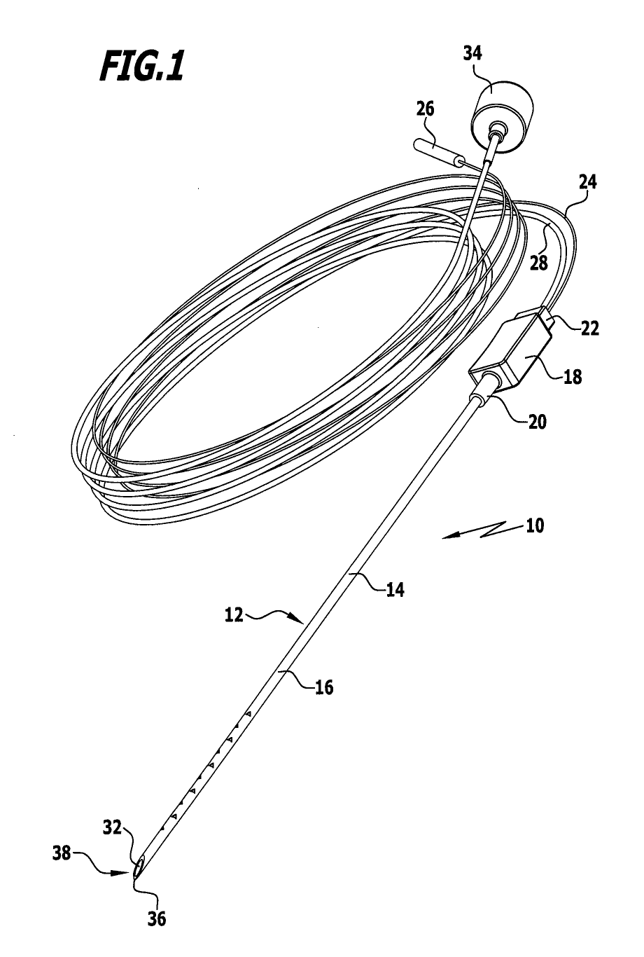

A medical instrument is designated by the reference numeral 10 in FIG. 1 by way of example and is in the form of a stimulation cannula for nerve blocks. It comprises an elongate instrument portion 12, which can be inserted into body tissue of patient, in the form of a hollow shaft 14, which forms a cannula 16.

At a proximal end of the instrument portion 12, the latter is coupled to a cuboid coupling part 18, which has a sleeve-shaped connecting piece 20 oriented in the distal direction, into which a proximal end of the instrument portion 12 is inserted. Orientated in the proximal direction and formed on the coupling part 18 is a receiver, which is not shown in more detail and into which a plug connector 22 can be inserted. The plug connector 22 makes it possible to produce an electrically conductive connection between a connection line 24 and the coupling part 18. The plug connector 22 and the coupling part 18 are formed in such a way that an electrically conductive connection can be produced between the connection line 24 and the cannula 16. A further plug connector 26, which can be connected to a current or voltage source, is provided at a proximal end of the connection line 24.

The cannula 16 defines, in its interior, a channel 32 extending coaxially with a longitudinal axis 30 of the shaft 14. The plug connector 22 that can be connected to the coupling part 18 is furthermore non-detachably coupled to a connection tube 28 which, when the plug connector 22 is coupled to the coupling part 18, has a fluid connection to the channel 32. Arranged on a proximal end of the connection tube 28 is a connector 34, which, for example, can be coupled to a syringe in order to guide fluids through the connection tube 28 and through the channel 32 to a distal end 36 and inject them into a patient's body.

The distal end 36 of the instrument portion 12 is in the form of a tip 38, which defines a tip plane, which is inclined relative to the longitudinal axis 30, specifically by means of an oval, annular end face 40, which surrounds an outlet opening 42 of the cannula 16. A distal end of the tip 38 may be in the form of a sharp or cutting protrusion 44 in order to facilitate the insertion of the instrument portion 12 into body tissue. A length 46 of the tip 38 extends between a distal end of the protrusion 44 and a proximal end of the end face 40.

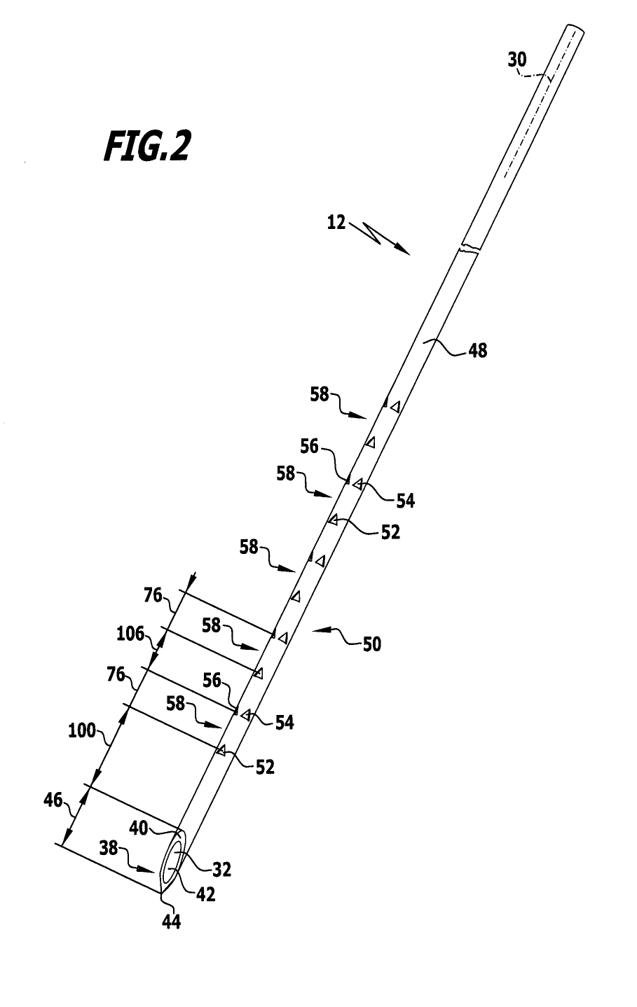

In order to be able to recognise the instrument portion 12 as well as possible on insertion into body tissue under ultrasonic observation, an outer surface 48 of the shaft 14 is provided with a surface structure designated as a whole by the reference numeral 50. The latter comprises a plurality of reflection elements 52, 54 and 56. Three respective reflection elements, namely the reflection elements 52, 54 and 56, form a substructure of the surface structure 50 in the form of a reflection element group 58. Therefore, a total of five reflection element groups 58, each comprising three respective reflection elements 52, 54 and 56, are provided in the instrument portion 12 shown in FIGS. 1 and 2.

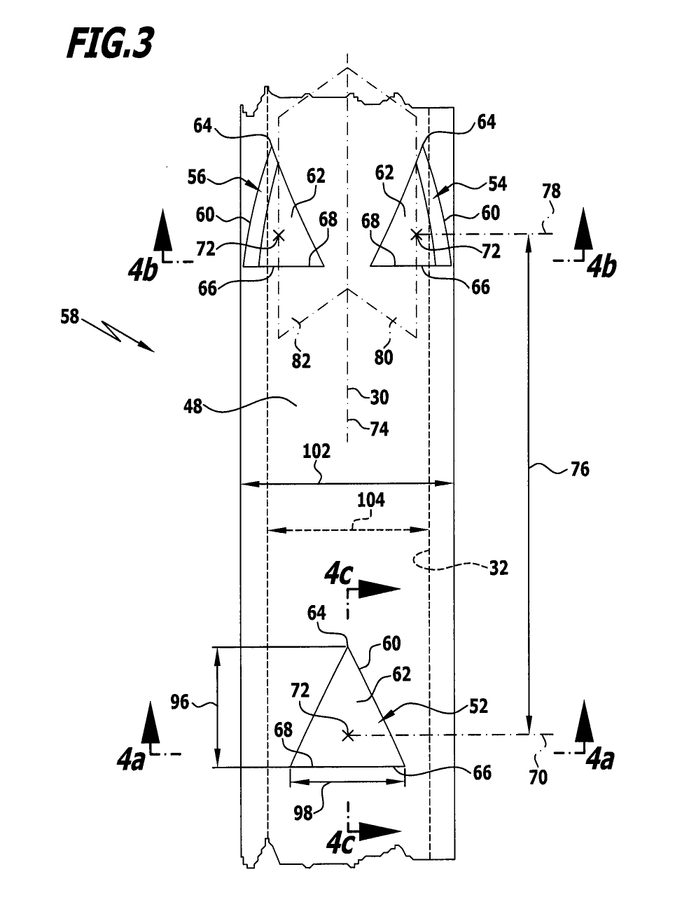

The reflection element groups 58 are all identically configured and will be described in more detail below in conjunction with FIG. 3.

All the three reflection elements 52, 54 and 56 are in the form of recesses 62, which, in each case, in plan view have the shape of an isosceles triangle. Each triangle 60 has a tip 64 pointing parallel to the longitudinal axis 30 in the proximal direction and an edge 66, which opposes the tip 64 and defines a transition region between an inner side face 68 of the recess 62 and the surface 48. The side face 68 defines a plane which runs transverse, in particular perpendicular, to the longitudinal axis 30. A first reflection element plane 70 defined by the reflection element 52 runs through a centroid 72 of the reflection element 52. Overall, the reflection element 52 is mirror-symmetrical with respect to a plane 74 of symmetry containing the longitudinal axis 30.

The reflection element 52 is arranged offset both relative to the reflection element 54 and to the reflection element 56 in the longitudinal direction, in other words parallel to the longitudinal axis 30 of the instrument portion 12. A spacing 76 is defined by the spacing between the first reflection element plane 70 and a second reflection element plane 78, which runs perpendicular to the longitudinal axis 30 and contains the centroids 72 of the reflection elements 54 and 56. The side faces 68 of the recesses 62 defining the reflection elements 54 and 56 define a plane running parallel to the second reflection element plane 78.

The reflection elements 54 and 56 are arranged and configured to be mirror-symmetrical with respect to the plane 74 of symmetry. Each reflection element 54 or 56 is also configured to be mirror-symmetrical to a plane 80 or 82 of symmetry, each of which contains the centroid of the respective reflection element 54 or 56 and the longitudinal axis 30. The planes 80 and 82 of symmetry are rotated with respect to the plane 74 of symmetry about an opening angle 84 in each case, which can have a value in a range of 25° to 65°.

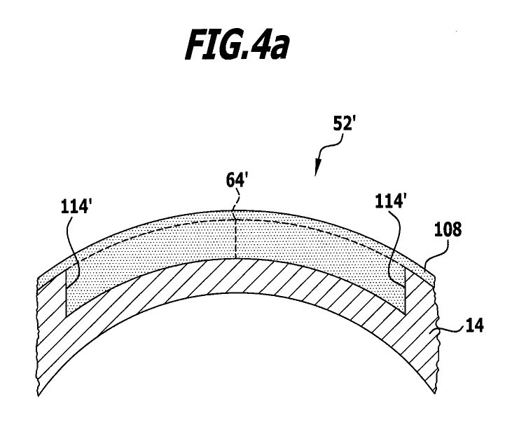

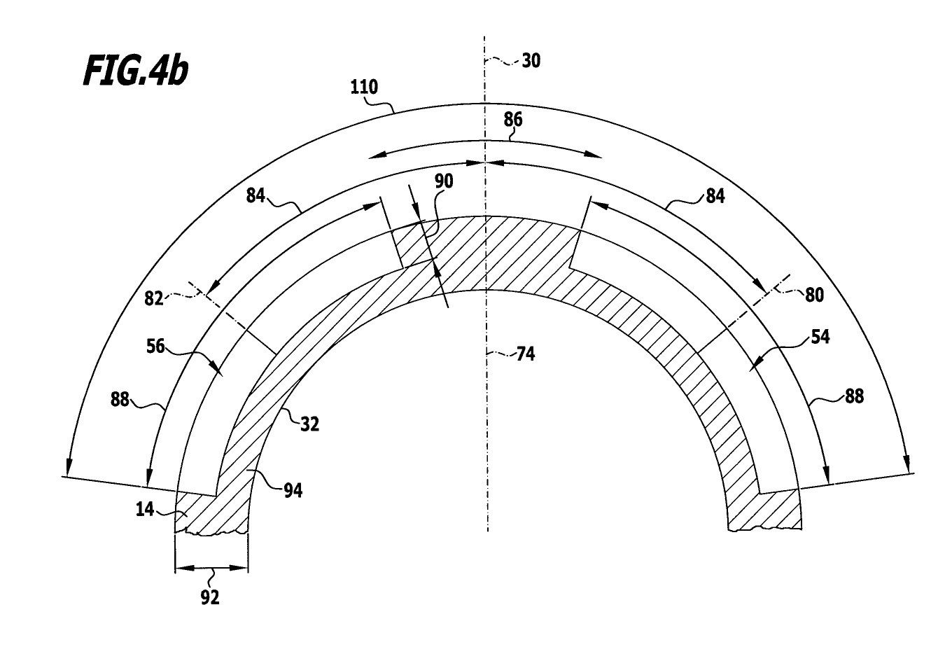

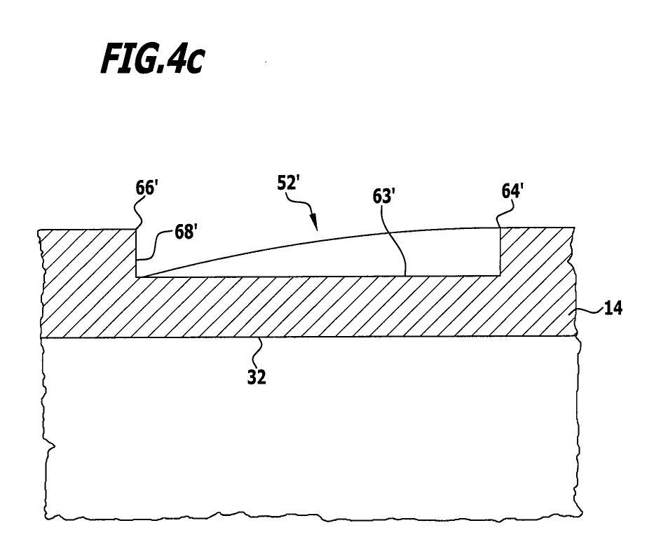

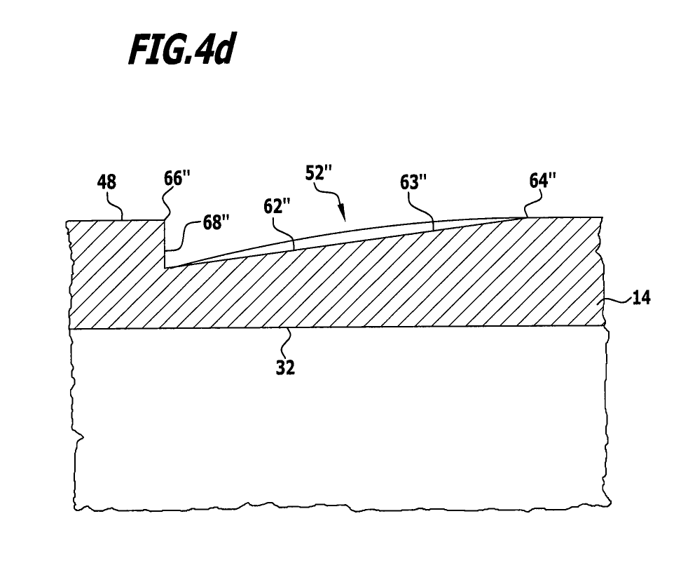

Both the reflection elements 54 and 56 and the reflection element 52 extend in the peripheral direction 86 in total over a reflection element angle range 88, which can have a value in a range from 5° to 80°. The reflection element angle range 88 advantageously has a value in the range from about 10° to about 70°. In the reflection angle range shown in FIG. 4b, the value thereof is about 50°. The surface structure 50 extends in total over an angle range 110, which is defined by the reflection elements 54 and 56. In particular, it has a value of a maximum of 180°, advantageously a maximum of 160°.

Each reflection element group 58 therefore comprises two reflection elements 54 and 56, which are arranged offset in the peripheral direction 86 transverse to the longitudinal axis 30 of the instrument portion 12, specifically by an angle, which corresponds to twice the opening angle 84.

A depth 90 of the recesses 82 is smaller than a thickness 92 of a wall 94 of the shaft 14. The depth 90 advantageously corresponds to about half the thickness 92. The depth 90 can, as in the embodiments shown in FIGS. 1 to 3 and 4a to 4c and 5 to 7, be constant over the entire area defined by the triangles 60. Alternatively, it would also be conceivable for a depth 90 of the recess 62″, in other words a spacing of a base 63″ of the recess 62 from the surface 48, proceeding from a maximum value, to decrease from the inner side face 68″ in the direction of the tip 64″, as shown schematically in FIG. 4d. The base 63″ is then inclined in relation to the longitudinal axis 30. The depth 90 to the tip 64 can optionally decrease to zero.

Each of the reflection elements 52, 54 and 56, parallel to the longitudinal axis 30, has a length 96, which corresponds to a height of the isosceles triangle 60. The length 96 advantageously has a value of 0.3±0.2 mm. A length 98 of the edge 66 advantageously also has a value of 0.3±0.2 mm. Optionally, the triangles 60 can also form equilateral triangles 60. A spacing 100 between a proximal end of the tip 38 and the first reflection element plane 70 of the reflection element group 58, which is closest to the tip 38, corresponds to about 1.3 to twice the length 46 of the tip 38. The spacing 100 is therefore greater than the length 46 of the tip 38. The spacing 76 corresponds approximately to 0.5 to 8 times the length 96. It may have a value in the range between 0.2 mm and 1.0 mm.

An external diameter 102 of the cannula 16 advantageously has a value in a range from 0.2 to 3.0 mm. An internal diameter 104 of the channel 32 advantageously has a value in the range from 0.1 mm to 2.5 mm. The thickness 92 of the wall 94 is advantageously in a range from 0.01 mm to 0.07 mm.

A reflection element group spacing 106 between adjacent reflection element groups 58 advantageously corresponds to the spacing 76. The reflection element group spacing 106 is defined by the spacing between a second reflection element plane 78 and a first reflection element plane 70 of the closest reflection element group 54 arranged on the proximal side.

The surface structure 50 comprises at least two reflection element groups 54, each with at least three and a maximum of nine reflection elements 52, 54 and 56. A total of eleven groups of three is particularly advantageous, in other words eleven reflection element groups 58, which therefore define eleven first reflection element planes 70 and eleven second reflection element planes 78 running parallel thereto.

The cannula 16 advantageously produced from metal is provided on the outside with an electrically insulating layer 108, which only leaves the end face 40 and the protrusion 44, optionally also only the protrusion 44, uncovered. The layer 108 is produced from a plastics material, advantageously from polytetrafluoroethylene (PTFE). The layer 108 can alternatively be formed from other plastics materials or from electrically insulating ceramic materials.

The reflection elements 52 and 54 or 56 can optionally differ with respect to their shape and size. In particular, it is conceivable for the reflection element 52 to be larger than the two reflection elements 54 and 56. Geometrical shapes differing from the triangle shape are also conceivable to form the reflection elements 52, 54 and 56, in particular polygons, for example quadrilaterals, pentagons or hexagons, as well as star-shaped, circular or oval reflection elements. Particularly advantageous configurations of the recesses 62 have edges 66 or inner side faces 68, which extend transverse to the longitudinal axis 30.

Alternatively, the reflection elements 52, 54 and 56 may also be in the form of protrusions. In particular, it is also conceivable, to configure the reflection elements 54 and 56, for example, in the form of protrusions and the reflection element 52 in the form of a recess 62 or accordingly vice versa.

Furthermore, the height and the depth 90 of at least one of the reflection elements 52, 54 and 56, for example, may vary in the peripheral direction 86 in relation to the longitudinal direction 30 of the instrument portion 12.

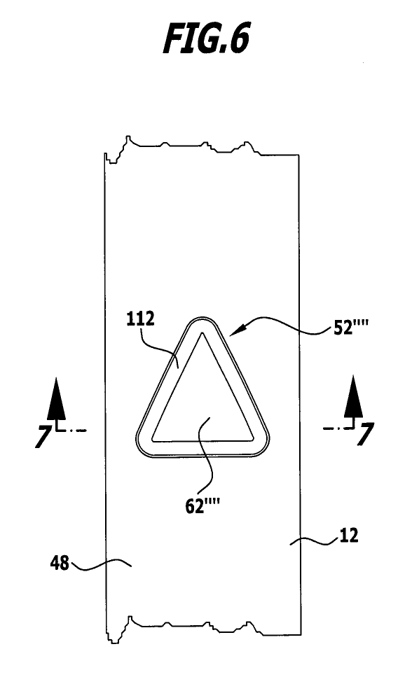

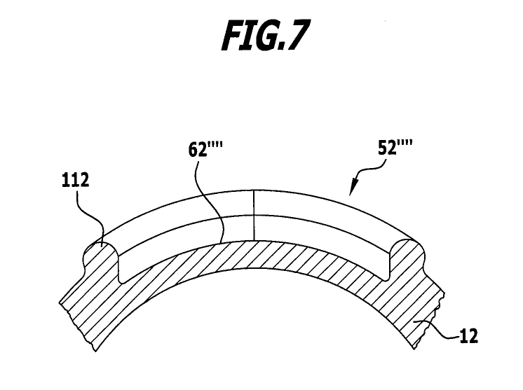

The reflection elements 52, 54 and 56 are advantageously produced by laser processing, in other words, the surface 48 of the cannula 16 is exposed to laser radiation of a suitable wavelength and intensity in order to evaporate the wall 94 to form the recesses 62. It is in particular possible with the laser processing of the cannula 16 to form a side boundary of a recess 62″″, shown schematically in FIGS. 6 and 7, in a bead-like manner, in other words in the form of a peripheral bead 112, which extends, at least in portions, advantageously wholly peripherally, slightly above the outer surface 48 of the instrument portion 12. A reflectivity of the surface structure 50 to ultrasound waves can be further increased by this special configuration of the recess 62″″.

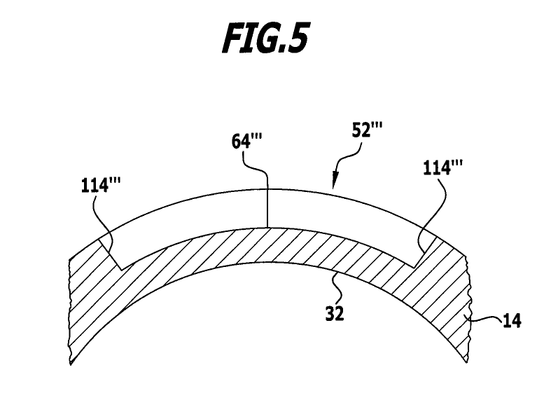

Inner side faces 68 and 114 of the recesses 62 may, in particular, be oriented perpendicular to the longitudinal axis 30 or such that they contain the longitudinal axis 30. Alternatively, side faces 114′ and a side face 68′, not shown, can be inclined in such a way that they intersect the longitudinal axis 30 at one point, in each case. A reflectivity of individual reflection elements 52′ or 52′″, as shown in FIGS. 4a and 5, can thus be additionally increased.

Under ultrasonic observation, the reflection elements 52, positioned on their own, are typically more clearly distinct and can be seen more clearly, and the reflection elements 54 and 56 together with the reflection elements 52 in each case form groups of three, which can be seen significantly better by the eye under ultrasonic observation than reflection elements 52 arranged one behind the other positioned on their own.

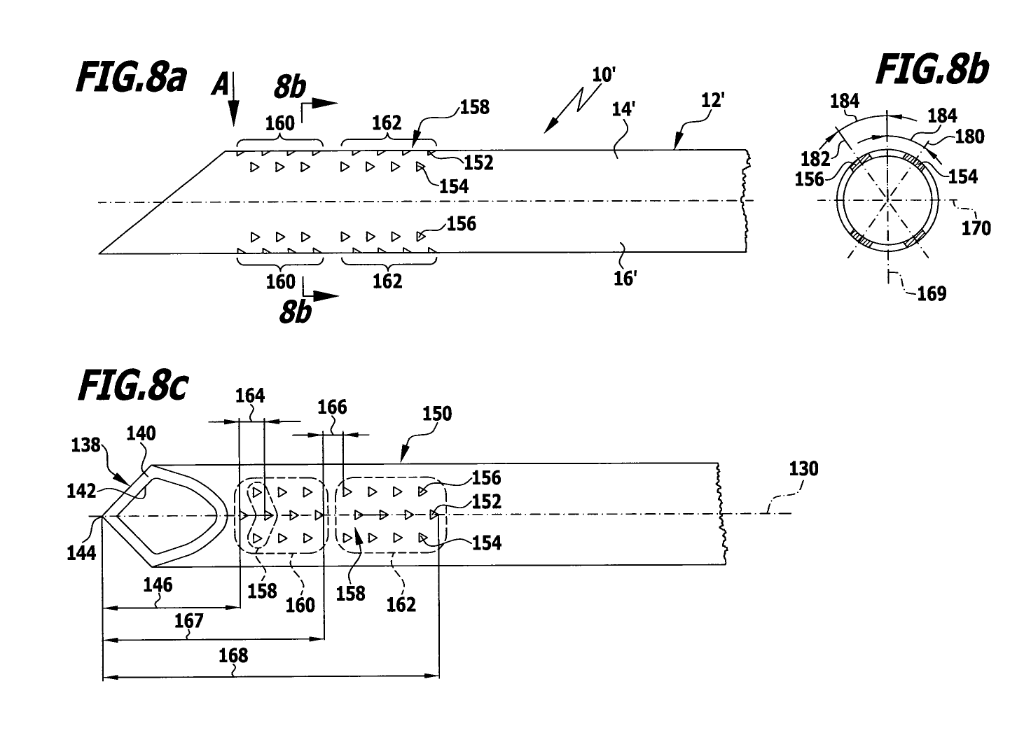

A distal end region of an elongate instrument portion 12′, which can be inserted in body tissue of a patient, of a further medical instrument 10′ is schematically shown in FIGS. 8a, 8b and 8c. The instrument portion 12′ is in the form of a hollow shaft 14′, which defines a cannula 16′.

The important difference from the stimulation cannula shown schematically in FIGS. 1 and 2 is the surface structure 150, which is modified compared to the surface structure 50. Said surface structure 150 in turn comprises a plurality of reflection elements 152, 154 and 156, which are arranged and formed in a defined manner regularly on the shaft 14′. The reflection elements 152, 154 and 156 may all be identically configured, for example like the reflection elements 52, 54 and 56, so that with regard to their specific configuration, reference can be made to the above description, in particular the statements in conjunction with FIGS. 1 to 4b. Alternatively, the reflection elements 152, 154 and 156 can also be in the form of the reflection elements 52′, 52″, 52′″ and 52″″ described in conjunction with FIGS. 4c to 7. In particular, it is also conceivable to use different embodiments of reflection elements to form the surface structures 50, 150, in other words, for example, a reflection element 152 in the form of the reflection element 52″″ and a reflection element 154 in the form of the reflection element 52′ or 52″.

Three reflection elements 152, 154 and 156 in each case form a substructure of the surface structure 150 in the form of a reflection element group 158. A total of 14 reflection element groups, with a reflection element 152, 154 and 156 in each case is therefore provided in the embodiment shown schematically in FIGS. 8a to 8c, one of the reflection element groups 158 additionally comprising a further reflection element 152.

In the embodiment shown schematically in FIGS. 8a to 8c, marking groups 160 and 162 are furthermore defined by the reflection element groups 158. The marking group 160 directly adjoins the tip 138 of the cannula 16′ on the proximal side. It comprises three reflection element groups 158 and a single reflection element 152. The reflection element groups 158 are mirror-symmetrical to a mirror plane 169 containing the longitudinal axis 130 of the shaft 14′, the reflection elements 152 being arranged in such a way that they are transformed by reflection on the mirror plane 169 to themselves and the reflection elements 154 and 156 are transformed by reflection to the other respective reflection element 156 or 154 in each case. The mirror plane 169 containing the longitudinal axis 130 can be transformed to itself by a second mirror plane 170 running perpendicular thereto.

The individual reflection element 152 is arranged directly adjacent to the tip 138. It can also be regarded as belonging to the most distal reflection element group 158, which then comprises a total four reflection elements. Reflection elements 152 following thereafter in the distal direction and arranged parallel to the longitudinal axis 130 are in each case arranged offset with respect to one another by the spacing 164. Identically formed reflection elements 154 and 156 are also in each case arranged offset with respect to one another by the spacing 164 parallel to the longitudinal axis 130 if they belong to the same marking group 160 or 162.

The marking groups 160 and 162 are slightly spaced apart or separated from one another, a proximal end of the most proximal reflection element 152 of the marking group 160 and the most distal end of the reflection elements 154 and 156 of the most distal reflection element group 158 of the marking group 162 being spaced apart from one another by the spacing 166.

A spacing 146 of the most distal reflection element from the distal end 144 of the tip 138 is only marginally greater than a length of the tip 138, so that, in other words, the surface structure 150 practically directly adjoins the tip 138 on the proximal side.

The spacing 146 is advantageously in a range from 1.5 to 2 mm and is advantageously 1.7 mm. A spacing 167 of a proximal end of the marking group 160 from the end 144 is advantageously in a range from 4.5 mm to 5.5 mm and is advantageously 5 mm. A spacing of a proximal end of the marking group 162 from the end 144 is advantageously in a range from 9 mm to 11 mm and is advantageously 10 mm. The spacing 144 is advantageously in a range from 0.8 mm to 1.2 mm and is advantageously 1 mm. The spacing 166 is advantageously in a range from 0.9 mm to 1.5 mm and is advantageously 1.2 mm.

The tip 138 is in principle formed analogously to the tip 38 and defines an outlet opening 142 with a substantially annular end face 140 inclined relative to the longitudinal axis 130.

The surface structure 150 comprises a total of four marking groups, namely two marking groups 160 and 162 in each case, which are arranged mirror-symmetrically with respect to the mirror plane 170. Alternatively, the marking groups 160 and 162 can also be transferred into one another by rotation through 180° about the longitudinal axis 130.

Schematically drawn in FIG. 8b is an opening angle 184, which is defined by the angle defined between the mirror plane 169 and the planes 180 and 182 of symmetry, which in each case contain the longitudinal axis 130, of the reflection elements 154 and 156. It is advantageously in a range from 25° to 65° and is advantageously 35°.

The surface structure 150 additionally facilitates the insertion of the cannula 16′ under ultrasonic observation as virtually on two sides of the cannula 16′ and specifically by the marking groups 160 and 162 arranged mirror-symmetrically with respect to the mirror plane 170, the surface structure 150 can be optimally detected under ultrasonic observation, practically regardless of a rotational position of the cannula 16′ about the longitudinal axis 130. The arrangement of the surface structure 150 in such a way that it almost directly adjoins the tip 138 on the proximal side, also makes it possible to detect the position of the tip 138 particularly precisely under ultrasonic observation. Optionally, the most distal reflection element 152 can even reach directly up to the end face 140 of the tip 138, so that, in this case, the spacing 146 coincides with the length of the tip 138.

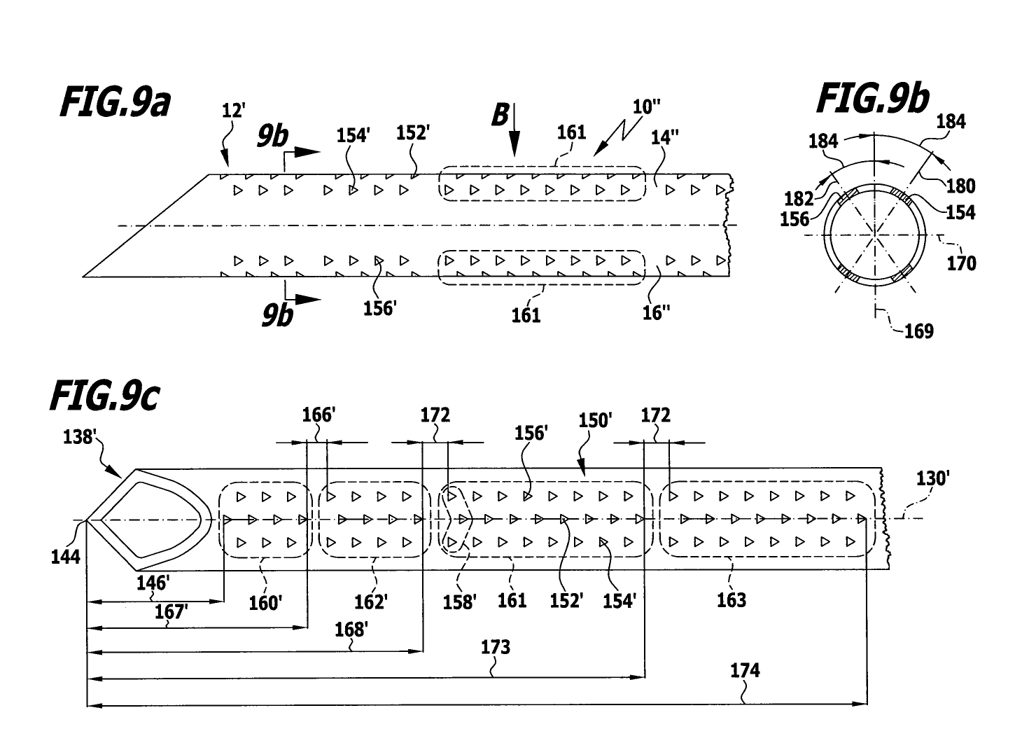

The embodiment of an instrument designated as a whole by the reference numeral 10″ shown schematically in FIGS. 9a to 9c is partially identical to the cannula 16′. All the symmetry considerations shown in conjunction with the instrument 10′ relating to the surface structure 150 also apply to the instrument 10″. There is also identity in the configuration of the tip 138′ to the tip 138. Identical elements of the instrument 10″ are therefore provided with the same reference numerals as in the instrument 10′ but with an apostrophe added thereafter.

The marking groups 160′ and 162′ are supplemented in the instrument 10″ to form the surface structure 150′ by two respective further marking groups 161 and 163. The two identical marking groups 161 are in each case mirror-symmetrical to the mirror plane 170. The marking group 161 adjoins the marking group 162′ on the proximal side and is separated from it by the spacing 172. Said spacing is advantageously in a range from 1.8 mm to 2.6 mm and is advantageously 2.2 mm. The marking groups 161 and 163 are in each case spaced apart from one another by the same spacing 172 and in each case comprise eight reflection element groups 158′. Each reflection element group 158′ in turn comprises, in each case, a reflection element 152′, 154′ and 156′, in other words a total of three reflection elements. A spacing 173 of a proximal end of the marking group 161 from the end 144′ is advantageously in a range from 18 mm to 22 mm and is advantageously 20 mm. A spacing 144 of a proximal end of the marking group 163 from the end 144′ is advantageously in a range from 28 mm to 32 mm and is advantageously 30 mm.

Optionally, the surface structure 150 of the instrument 10′ can also be only supplemented by one or two marking groups 161 formed symmetrically to the mirror plane 170. Furthermore, the surface structure 150′ can also be supplemented by further marking groups 160′, 162′, 161 and/or 163, which then adjoin the marking group 163 on the proximal side and can be spaced apart therefrom, for example by the same spacing 172.

The number of reflection elements belonging to a reflection element group 158 or 158′ may optionally also be greater than three, but there are advantageously no more than nine reflection elements defining a reflection element group 158 or 158′. The number of reflection element groups 158 or 158′ for the respective marking groups 160, 162, 160′, 162′, 161 and 163 may also vary and differ from the respective number in the schematically shown embodiments of the cannulas 16′ and 16″ and therefore also vary as desired.

The longer the cannula 16′ or 16″, the more marking groups can be provided on the respective instrument portion 12′, depending on the purpose of use of the cannula 16′ or 16″. The reflection elements of the instruments 10′ and 10″ are advantageously also formed by laser processing on the shaft 14′ or 14″.

FIGS. 8a to 9c show purely schematically the configuration of alternative surface structures 150 and 150′. Moreover, the structure of the cannulas 16′ or 16″ may correspond to the structure of the cannula 16, in particular a connection of the respective cannula 16′ or 16″ to a connection line 24.

1. A medical instrument with at least one instrument portion, which can be inserted into body tissue of a patient and has a surface structure reflecting ultrasound waves, the surface structure comprising a plurality of reflection elements, wherein at least three and a maximum of nine reflection elements, which are arranged in a defined manner relative to one another, define a reflection element group and wherein the surface structure comprises at least two reflection element groups.

2. The medical instrument according to claim 1, wherein each reflection element group comprises at least two reflection elements, which are arranged offset with respect to one another in the longitudinal direction of the instrument portion.

3. The medical instrument according to claim 2, wherein the at least two reflection elements arranged offset with respect to one another in the longitudinal direction of the instrument portion are identically configured.

4. The medical instrument according to claim 2, wherein a spacing in the longitudinal direction between two reflection elements arranged offset with respect to one another in the longitudinal direction of the instrument portion, of a reflection element group corresponds to Y-times a length of the smaller of the reflection elements in the longitudinal direction and wherein Y has a value in the range of 0.5 to 8.

5. The medical instrument according to claim 1, wherein a reflection element group spacing in the longitudinal direction between two reflection element groups arranged offset with respect to one another in the longitudinal direction of the instrument portion corresponds to Z-times a length of the smallest of the reflection elements in the longitudinal direction and wherein Z has a value in the range from 0.5 to 8.

6. The medical instrument according to claim 1, wherein the surface structure as a whole extends over an angle range of a maximum of 180° in the peripheral direction in relation to a longitudinal axis of the instrument portion.

7. The medical instrument according to claim 1, wherein the reflection elements in each case extend over a reflection element angle range of about 5° to about 80° in the peripheral direction based on a longitudinal axis of the instrument portion.

8. The medical instrument according to claim 1, wherein the reflection elements are in the form of recesses and/or protrusions.

9. The medical instrument according to claim 8, wherein the shaft comprises a wall and wherein a height and/or a depth of the reflection elements in relation to a longitudinal axis of the instrument portion is smaller than a thickness of the wall.

10. The medical instrument according to claim 9, wherein the height and/or the depth of the reflection elements is at most half as great as the thickness of the wall.

11. The medical instrument according to claim 9, wherein the height or the depth of at least one of the reflection elements parallel to the longitudinal direction of the instrument portion varies.

12. The medical instrument according to claim 9, wherein the height or the depth of at least one of the reflection elements varies in the peripheral direction in relation to the longitudinal direction of the instrument portion.

13. The medical instrument according to claim 1, wherein at least one reflection element of each reflection element group has an edge or side face extending transverse to a longitudinal direction of the instrument portion.

14. The medical instrument according to claim 1, wherein each reflection element group defines at least two reflection element planes spaced apart from one another in the longitudinal direction.

15. The medical instrument according to claim 8, wherein a side boundary of a recess is configured in the manner of a bead and projects slightly, at least in portions, over an outer surface of the at least one instrument portion.

16. The medical instrument according to claim 1, wherein at least one reflection element of a reflection element group is in the form of a triangle.

17. The medical instrument according to claim 1, wherein the number of reflection element groups is in a range from 3 to 25.

18. The medical instrument according to claim 1, wherein the reflection elements are formed by laser processing of the at least one instrument portion.

19. The medical instrument according to claim 1, wherein the at least one instrument portion has a distal end, which is in the form of a tip.

20. The medical instrument according to claim 19, wherein a spacing between a proximal end of the tip and a distal end of a reflection element group provided adjacent to the tip corresponds to X-times the length of the tip and wherein X is in a range from 0.5 to 5.

21. The medical instrument according to claim 1, wherein an instrument portion in the form of a cannula.

22. The medical instrument according to claim 1, wherein the at least one instrument portion has an outer surface, which comprises the surface structure reflecting the ultrasound waves and which is provided with an electrically insulating layer.

23. The medical instrument according to claim 22, wherein the insulation material is a plastics material or a ceramic.

24. The medical instrument according to claim 23, wherein the plastics material is polytetrafluoroethylene (PTFE).

25. The medical instrument according to claim 1, wherein the instrument comprises an electrical connection device to connect the instrument to a current or voltage source.
